(19) United States
(12) Patent Application Publication
(10) Pub. No.: US 2013/0123345 A1
(43) Pub. Date: May 16, 2013

(54) METHOD OF TREATING A VIRAL INFECTION DYSFUNCTION BY DISRUPTING AN ADENOSINE RECEPTOR PATHWAY

(71) Applicant: Ian C. Davis, Hilliard, OH (US)

(72) Inventor: Ian C. Davis, Hilliard, OH (US)

(73) Assignee: THE OHIO STATE UNIVERSITY, Columbus, OH (US)

(21) Appl. No.: 13/811,732

(22) PCT Filed: Jul. 22, 2011
(86) PCT No.: PCT/US11/45035

§ 371 Date: Jan. 23, 2013

Related U.S. Application Data
(60) Provisional application No. 61/366,986, filed on Jul. 23, 2010.

(51) Int. Cl. A61K 31/7076; A61K 31/522; A61K 31/277

(52) U.S. Cl. CPC A61K 31/7076; A61K 31/277; A61K 31/522
USPC 514/44 A; 514/521; 514/47; 514/263.34; 514/564; 514/406; 514/510; 514/43; 514/218; 514/354; 514/339; 514/651

(57) ABSTRACT

Described herein is a method of treating a viral infection such as an influenza infection, in a subject comprising administering an effective amount of a pharmaceutical composition to disrupt a adenosine receptor pathway, such as the Aradenosine receptor pathway, in a subject. The adenosine receptor pathway includes the steps of 1) producing the adenosine precursor adenosine triphosphate (ATP), 2) releasing ATP into the extracel lular space, 3) enzymatic conversion of ATP to adenosine, 4) activation of the adenosine receptor and the adenosine receptor cascade, and 5) clearance of adenosine from the extracellular space by degradation or uptake into a cell. The method includes affecting at least one of these steps so as to decrease the activation of the adenosine receptor pathway. This may be accomplished by decreasing the production, release, or conversion of ATP to adenosine, decreasing the expression of the adenosine receptor, antagonizing adenosine receptor activation, and/or increasing adenosine clearance.

### CROSS-REFERENCE TO RELATED APPLICATIONS

This application claims the benefit of and priority to prior filed co-pending Provisional Application Ser. No. 61/366,986, filed Jul. 23, 2010, which is expressly incorporated herein by reference in its entirety.

### FIELD

The present invention relates generally to a treatment for a viral infection and more particularly to a treatment of the pulmonary, cardiovascular, and renal clinical signs, and symptoms of a viral infection, such as influenza infection, that are mediated by adenosine receptors.

### BACKGROUND

Many viral infections, such as influenza, are highly contagious and deadly. For example, despite vaccination and use of antiviral drugs, seasonal influenza causes in excess of 36,000 deaths per year in the United States. Moreover, the threat of pandemic influenza outbreaks, similar to those seen in the 20^{th} century, threatens to cause devastating loss of life.

Vaccines and antiviral drugs are designed to target the virus itself. However, many viruses, such as the influenza virus, mutate rapidly necessitating annual vaccine reformulations and raising concerns about resistance to antiviral drugs. Thus, new therapeutic approaches are needed that target the consequences of infection by the virus in the human host, instead of targeting the virus itself. Targeting the consequences of infection, rather than targeting the virus, has the unique advantage that it will avoid the issue of the virus developing resistance to the treatment.

Virus mediated lung damage, such as caused by the influenza virus, can lead to hypoxemia and pneumonia and is a cause of the high mortality in humans associated with viral infection. Viral infections can also cause suppression of cardiac and renal function. A therapeutic approach that blocks or decreases virus mediated lung damage, cardiac dysfunction, or renal failure could result in improved clinical outcomes for patients by allowing them to survive the initial viral insult while the infection runs its course. Mechanisms underlying lung, heart, and kidney dysfunction in viral infections such as influenza remain poorly defined.

### SUMMARY

Severe viral pneumonia, such as influenza pneumonia, results in lung dysfunction consistent with current clinicopathologic definitions of acute lung injury. Lung injury may also be accompanied by cardiac or renal dysfunction or outright failure in virus-infected patients. Adenosine, a chemical messenger, plays a proinflammatory role in acute lung injury pathogenesis, and also has effects on cardiac and renal function which tend to promote cardiac overload. Influenza infection results in increased adenosine generation and adenosine receptor activation in the lung, and also detrimental effects on the function of the heart and kidneys. Detrimental effects of influenza infection for the heart and kidneys may be mediated either by adenosine “spillover” into the systemic circulation from the influenza-infected lung, or as a consequence of increased local generation of adenosine from plasma ATP as a response to hypoxemia (itself a consequence of influenza infection and associated lung dysfunction). Disruption of the adenosine receptor pathway provides a new therapeutic strategy for decreasing acute lung injury, cardiac suppression, and acute renal failure mediated by a viral infection, such as infection with the influenza virus or other viruses that affect adenosine pathways in a subject. This strategy improves the outcome of a subject without directly targeting the virus and thereby does not increase the risk of viral mutations resulting in drug resistant strains. Accordingly, described herein is a method of treating a viral infection in a subject comprising administering an effective amount of a pharmaceutical composition to disrupt the adenosine receptor pathway in the subject. The adenosine receptor pathway includes the steps of 1) producing the adenosine precursor adenosine triphosphate (ATP), 2) releasing ATP into the extracellular space, 3) enzymatic conversion of ATP to adenosine, 4) expression of the adenosine receptor mRNA and protein from its encoding gene in the target cell, 5) activation of the adenosine receptor, and 6) clearance of adenosine from the extracellular space by degradation or uptake into a cell. The method includes affecting at least one of these steps so as to decrease the activation of the adenosine receptor pathway. This may be accomplished by decreasing the production, release, or conversion of ATP to adenosine, antagonizing adenosine receptor gene and/or protein expression, antagonizing adenosine receptor activation, and/or increasing adenosine clearance.

### BRIEF DESCRIPTION OF THE DRAWINGS

FIG. 1 is an illustration of some steps of the adenosine receptor pathway.

FIG. 2A is a graph illustrating the effect of influenza infection on ATP levels in lung tissue.

FIG. 2B is a graph illustrating the effect of influenza infection on markers of epithelial cell death in lung tissue.

FIG. 2C is a graph illustrating the reversal of influenza-induced suppression of alveolar clearance by pharmacological disruption of ATP synthesis or release.

FIG. 3A is a graph illustrating a timeline of influenza-mediated decrease in alveolar fluid clearance.

FIG. 3B is a graph illustrating a timeline of influenza-mediated decrease in pulmonary gas exchange.

FIG. 3C is a graph illustrating a timeline of influenza-mediated increase in total lung resistance.

FIG. 3D is a graph illustrating a timeline of influenza-mediated decrease in lung compliance.

FIG. 4A is a graph illustrating that inhibition of CD73 had no effect on influenza-induced weight loss.

FIG. 4B is a graph illustrating that inhibition of CD73 significantly delayed influenza-induced mortality.

FIG. 4C is a graph illustrating that inhibition of CD73 significantly delayed the onset of influenza-induced peripheral hypoxemia.

FIG. 5A is a graph illustrating that the onset of influenza-induced peripheral hypoxemia is significantly delayed and attenuated in adora1^{-/-} mice.

FIG. 5B is a graph illustrating that influenza-mediated lung water content is significantly decreased in adora1^{-/-} mice.

FIG. 5C is a graph illustrating that inflammatory cell infiltration into BALF is significantly decreased in adora1^{-/-} mice.

FIG. 5D is a graph illustrating that influenza-induced increases in airway resistance at 6 d.p.i. are absent in adora1^{-/-} mice.

FIG. 5E is a graph illustrating that influenza-induced increases in airway hyperresponsiveness to the bronchoconstrictor methacholine at 2 d.p.i. are absent in adora1^{-/-} mice.

FIG. 5F is a graph illustrating that influenza-induced decreases in static lung compliance at 6 d.p.i. are absent in adora1^{-/-} mice.

FIG. 6A is a graph illustrating that influenza increases adora1 gene expression in both whole lung and alveolar type II cells.

FIG. 6B is a graph illustrating that A1-adenosine receptor protein is preferentially expressed on the surface of influenza-infected alveolar type II cells.

FIG. 7A is a graph illustrating that antagonism of the A1-adenosine receptor significantly delayed influenza-induced mortality.

FIG. 7B is a graph illustrating that antagonism of the A1-adenosine receptor significantly delayed the onset of influenza-induced peripheral hypoxemia.

FIG. 7C is a graph illustrating that antagonism of the A1-adenosine receptor significantly decreased influenza-mediated lung water content.

FIG. 8A is a graph illustrating that influenza infection resulted in severe bradycardia (low heart rate), and that bradycardia is absent in influenza-infected adora1^{-/-} mice.

FIG. 8B is a graph illustrating that influenza infection resulted in severe bradycardia (low heart rate), and that antagonism of the A1-adenosine receptor significantly increased heart rate in influenza-infected mice.

### DETAILED DESCRIPTION

An aspect of the invention is a method of treating a viral infection, such as an infection with all strains of influenza A and B viruses, including H5N1 “avian flu” and H1N1 swine-origin “swine flu” viruses, in a subject by administering an effective amount of a pharmacological composition to disrupt the adenosine receptor pathway. Other viral infections that affect the adenosine receptor pathway may be treated with the inventive method, such as Paramyxoviridae (e.g. respiratory syncytial virus, Hendra virus, and Nipah virus), Togaviridae (e.g., rubella virus), Hantaviridae (e.g., Sin Nombre virus), Rhinoviridae, Coronoviridae, Herpesviridae (e.g., Epstein Barr virus, and cytomegalovirus), Adenoviridae, and Filoviridae. Another aspect of the invention is a method of treating virus-mediated pulmonary damage in a subject by administering an effective amount of a pharmaceutical composition to disrupt the adenosine receptor pathway in the lung of the subject. Another aspect of the invention is a method of treating virus-mediated cardiac and/or renal dysfunction in a subject by administering an effective amount of a pharmaceutical composition to disrupt the adenosine receptor pathway in the heart and/or kidneys of the subject.

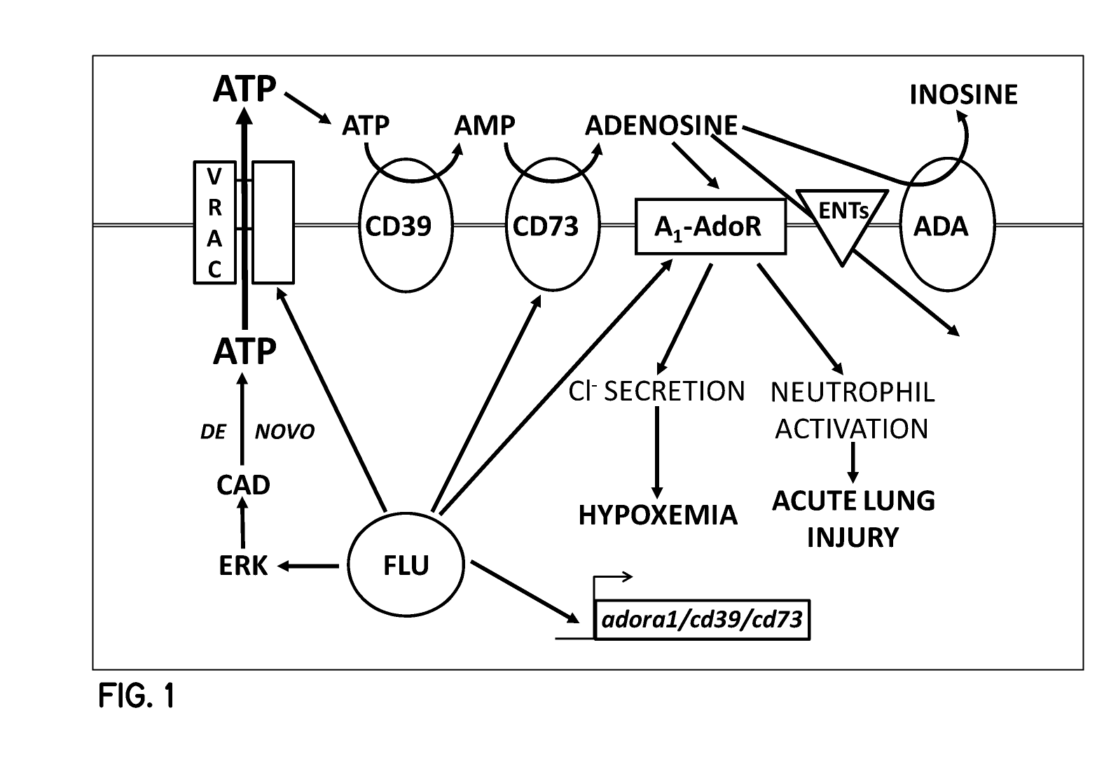

The adenosine receptor pathway includes multiple steps that may be disrupted to treat viral infection symptomology. Referring now to FIG. 1, these steps include the synthesis of the adenosine precursor adenosine triphosphate (ATP), release of ATP from synthesizing cells, conversion of ATP to adenosine, expression of the adenosine receptor by the target cell, activation of the adenosine receptor, and clearance of adenosine from the extracellular space, which further includes enzymatic degradation of adenosine and adenosine transport into a nearby cell.

Without being bound to any particular theory, viral infection, such as influenza infection, activates cytoplasmic extracellular signal-regulated kinase (ERK) in alveolar epithelial type II cells (ATII cells) which stimulates de novo nucleotide synthesis, such as the synthesis of adenosine triphosphate (ATP). Disrupting the activation of the signaling pathway that stimulates ATP production or, in the alternative, direct inhibition of the enzymes responsible for the production of ATP decreases cellular ATP concentrations. Decreasing cellular ATP concentrations decreases the amount of ATP available for release into the extracellular space available for conversion to adenosine and thus decreases activation of the adenosine receptor cascade.

Exemplary compounds that disrupt the de novo synthesis of ATP include A77-1726 (also referred to as teriflunomide), a pyrimidine synthesis inhibitor, and U0126 (1,4-diamino-2,3-dicyano-1,4-bis[2-aminophenylthio] butadiene), an ERK MAP kinase inhibitor.

ATP synthesized in the cell is actively released from the cell via volume-regulated anion channels (VRACs), whose opening is facilitated by virus-mediated Rho kinase activation. Blocking the Rho kinase or VRAC activity thus blocks the release of ATP thereby decreasing the amount of ATP available in the extracellular space for conversion to adenosine which decreases the activity of the adenosine receptor cascade.

Exemplary compounds that disrupt Rho kinase include H-1152 ((S)-(+)-2-Methyl-1-[(4-methyl-5-isoquinolinyl)sulfonyl]homopiperazine), NNU (N-(4-Pyridyl)-N′-(2,4,6-trichlorophenyl)urea), Rockout (3-(4-Pyridyl)-1H-indole), and pyrazol carboxamide (N-(4-(1H-pyrazol-4-yl)phenyl)-2,3-dihydrobenzo[b][1,4]dioxine-2-carboxamide). Exemplary compounds that disrupt VRACs include fluoxetine, clomiphene, verapamil, NPPB (5-nitro-2-(3-phenylpropylamino) benzoic acid), R(+)-IAA 94 (R(+)-([6,7-dichloro-2-cyclopentyl-2,3-dihydro-2-methyl-1-oxo-1H-inden-5-yl]-oxy)acetic acid 94), and tamoxifen.

ATP released into the extracellular space is sequentially converted to adenosine by CD39 and CD73. CD39 catabolizes ATP to adenosine monophosphate (AMP) which is converted to adenosine by CD73. CD73 activity, which may be increased during influenza infection, is the rate-limiting step for adenosine formation. Increased cd73 gene and CD73 protein expression occurs in response to activation of hypoxia-inducible factor-1α (HIF-1α) in cells experiencing influenza-related hypoxia. Inhibition of CD39 expression and/or enzymatic activity will decrease the amount of AMP available for conversion to adenosine by CD73 and therefore decrease the amount of adenosine available to activate the adenosine receptor cascade. Likewise, inhibition of CD73 expression and/or enzymatic activity will similarly decrease adenosine availability for receptor activation.

Exemplary compounds that decrease CD39 activity include polyoxometalate-1 (POM-1), ARL67156, small inhibitory RNAs directed against CD39 mRNA, microRNAs directed against CD39 mRNA, and vector-mediated or other constructs designed to specifically induce inactivation of cd39 gene transcription and/or translation. Exemplary compounds that decrease CD73 activity include APCP (5′-(α,β-methylene)diphosphate). Exemplary compounds that inhibit CD73 expression include inhibitors of HIF-1α, small inhibitory RNAs directed against CD73 mRNA, microRNAs directed against CD73 mRNA, and vector-mediated or other constructs designed to specifically induce inactivation of cd73 gene transcription and translation.

Binding of adenosine to adenosine receptors, such as the A₁-adenosine receptor (A₁-AdoR) on lung epithelial cells stimulates chloride ion (Cl⁻) and fluid secretion into airspaces, contributing to development of hypoxemia. In addition, adenosine activation of A₁-AdoR on neutrophils results in their activation to contribute to acute lung injury in severe influenza. Binding of adenosine to A₁-AdoR on cardiac pacemaker cells induces bradycardia (reduced heart rate) and reduced responsiveness to the positive inotropic and chronotropic effects of β-agonists. Binding of adenosine to A₁-AdoR on cells in the kidney reduces glomerular filtration, inhibition of renin release, and increased tubular reabsorption of Na⁺. Together, these effects induce volume retention and cardiac overload. Thus, adenosine receptors, such as the A₁-AdoR, are promising potential targets of viral infection therapy, such as treatment of the adenosine mediated pulmonary, cardiac, and renal symptomology associated with viral infections, such as influenza infection. Viral infection may also increase A₁-AdoR gene and protein expression by uninfected and/or virus-infected target cells via activation of the transcription factor NE-κB. Thus, inhibition of NF-κB activity and/or A₁-AdoR gene transcription, translation, and protein expression will similarly decrease A₁-AdoR availability on target cells for activation by adenosine generated in response to virus infection.

Exemplary non-specific adenosine receptor antagonists include caffeine and theophylline. While non-specific adenosine receptor antagonists may be useful in the inventive method when administered at the appropriate dose and route of administration, non-specific antagonists such as caffeine are more likely than specific A₁-AdoR antagonists to have concomitant detrimental effects via activation of other adenosine receptor subtypes, which reduces their therapeutic value, particularly when not administered directly to the targeted tissue such as the lungs. Some of these side-effects may be particularly detrimental in persons with lung injury coupled to cardiovascular or renal dysfunction. For example, caffeine causes increased heart cardiac output, which increases the O₂ demand of the heart, and caffeine also causes diuresis, which similarly increases O₂ demands of kidney. Thus, caffeine consumption in a hypoxemic subject can make both organs more susceptible to injury. For example, caffeine is generally orally ingested in relatively high doses (tens of milligrams per kilogram body weight per day), which can lead to these detrimental effects. Thus, orally ingested non-specific adenosine receptor antagonists are not within the scope of the invention. However, for example, the non-specific antagonists can be effective if administered via inhalation allowing direct contact with an infected lung.

Exemplary selective A₁-AdoR antagonists include L-97-1 (available from Endacea Inc.), SLV320 (available from Solvay Pharmaceuticals), rolofylline (available from Kyowa Hakko, Japan), 8-cyclopentyl-1,3-dipropylxanthine (DPCPX), and cyclopentyltheophylline. Some of these adenosine receptor antagonists, such as L-97-1, SLV320, and rolofylline, are currently available for indications unrelated to viral infections such as influenza, and appear to be safe and well-tolerated in humans. Exemplary NE-κB inhibitors include PDTC and BAY 11-7082. Exemplary compounds that reduce A₁-AdoR expression include small inhibitory RNAs directed against A₁-AdoR mRNA, microRNAs directed against A₁-AdoR mRNA, and vector-mediated or other constructs designed to specifically induce inactivation of A₁-AdoR (adora1) gene transcription and translation.

Increasing the clearance of adenosine from the extracellular space decreases the availability of adenosine to activate adenosine receptors. One mechanism for removing adenosine from the extracellular space includes adenosine degradation to inosine by adenosine deaminase (ADA). Another mechanism involves increasing the uptake of adenosine into a cell, such as by the equilibrative nucleoside transporter (ENT).

Exemplary compositions that increase adenosine deaminase activity include 2′-deoxycoformycin and 2-N-methyl-2,4-diazacycloheptanone. Exemplary compositions that increase ENT activity include compounds that activate protein kinase C, such as PMA (phorbol 12-myristate 13-acetate) or those that inhibit hypoxia inducible factor-1 (HIF-1) activity, such as YC-1 (3-(5′-hydroxymethyl-2′-furyl)-1-benzyl indazole) and PX-478.

Thus, treating dysfunctions associated with influenza infection, such as the pulmonary, cardiac, and renal dysfunctions is accomplished by administering an effective amount of a pharmaceutical composition that affects any of the above described steps in an adenosine receptor pathway, such as the A₁-AdoR. These compounds may generally be administered over a dose range from about 1 micromole/kg/day to about 1 millimole/kg/day, and in any event the dose is sufficient to disrupt the adenosine receptor pathway, especially the A₁-AdoR pathway, at levels sufficient to treat a pulmonary, cardiac, and/or renal dysfunction in a subject. Those skilled in the art can determine the appropriate level of dosing needed for each composition. As discussed in greater detail below, the dosing may be affected by the route of administration used for the compositions.

The inventive methods may be useful for the treatment of dysfunctions resulting in symptomology sufficient to warrant consultation of a healthcare professional, particularly a physician, or attendance at or referral to an Emergency Room. For example, a 10-20% alteration in lung or heart function, and a 50% decrease in renal function from that of a healthy human are exemplary ranges of dysfunction that may require treatment. The inventive methods result in a reduction in symptomology or clinically-determined organ dysfunction of sufficient significance as to allow release from physician care.

In one embodiment, pulmonary dysfunction may be characterized by a decrease in lung function as may be determined by, for example, mucosal membrane cyanosis, hyperventilation, hypoventialtion, altered respiratory effort; hemoglobin O2 saturation; arterial blood gases (PaO2, PaCO2, electrolytes, anion gap, P:F ratio), chest x-ray, CT scan, MRI, or PET scan to quantitate pulmonary edema, technetium imaging to quantitate lung clearance rate, pulmonary arterial wedge pressure, measurement of lung mechanics (FEV1, total lung capacity, P-V loop), BAL fluid inflammatory markers (inflammatory cell infiltrates, protein, LDH, cytokines, chemokines, and RONS), exhaled breath condensate inflammatory markers, and any other clinical tests known to those skilled in the art.

Cardiac dysfunction may be characterized by a decrease in cardiac function as may be determined by, for example, alterations in blood pressure, pulse/heart rate, ECG tracings, abnormalities of shape, size or function (ejection fraction, stroke volume, fill time) detected by ultrasound or other imaging modalities, plasma indices of cardiac damage such as troponin-T and lactate dehydrogenase, and any other clinical tests known to those skilled in the art.

Renal dysfunction may be characterized by a decrease in renal function as may be determined by, for example, changes in urine volume, tonicity, and/or composition, plasma assays of renal function such as BUN and creatinine, and renal function tests such as inulin administration to measure glomerular filtration rate, and any other clinical tests known to those skilled in the art.

The compositions can be administered in vivo in a pharmaceutically acceptable carrier. By “pharmaceutically acceptable” is meant a material that is not biologically or otherwise undesirable. Thus, the material may be administered to a subject, without causing undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art. The materials may be in solution, suspension (for example, incorporated into microparticles, liposomes, or cells). These may be targeted to a particular cell type via antibodies, receptors, or receptor ligands.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carriers include, but are not limited to, saline, Ringer's solution and dextrose solution. The pH of the solution is in a pharmaceutically acceptable range, preferably from about 5 to about 8.5, and more preferably from about 7.8 to about 8.2. Further carriers include sustained release preparations such as semipermeable matrices of solid hydrophobic polymers containing the pharmaceutical composition, which matrices are in the form of shaped articles, e.g., films, liposomes or microparticles. It will be apparent to those persons skilled in the art that certain carriers may be more preferable depending upon, for instance, the route of administration and concentration of composition being administered. For example, it is within the skill in the art to choose a particular carrier suitable for inhalational and/or intranasal administration, or for compositions suitable for topical administration to a pulmonary epithelial cell or for introduction to the body by injection, ingestion, or transdermally.

The pharmaceutical compositions may also include thickeners, diluents, buffers, preservatives, surface active agents, and the like in addition to the compositions and carriers. The compositions may also include one or more active ingredients such as antimicrobial agents, anti-inflammatory agents, anesthetics, and the like.

The disclosed compositions are suitable for topical administration to a pulmonary epithelial cell or to a plurality of pulmonary epithelial cells of a subject. Thus, the compositions comprising an effective amount of a disruptor of an adenosine receptor pathway are optionally suitable for administration via inhalation, (i.e., the composition is an inhalant). Further, the compositions are optionally aerosolized. And, further still, the compositions are optionally nebulized. Administration of the compositions by inhalation can be through the nose or mouth via delivery by a spraying or droplet mechanism. Delivery can also be directly to any area of the respiratory system (e.g., lungs) via intubation. Optionally, the pulmonary epithelial cell to which a composition is administered is located in the nasal cavity, nasal passage, nasopharynx, pharynx, trachea, bronchi, bronchiole, or alveoli of the subject. Optionally, the pulmonary epithelial cell to which a composition is administered is a bronchoalveolar epithelial cell. Moreover, if the compositions are administered to a plurality of pulmonary epithelial cells, the cells may be optionally located in any or all of the above anatomic locations, or in a combination of such locations.

Topical administration to a pulmonary epithelial cell accordingly may be made by pulmonary delivery through nebulization, aerosolization, or direct lung instillation. Thus, compositions suitable for topical administration to a pulmonary epithelial cell in a subject include compositions suitable for inhalant administration, for example as a nebulized or aerosolized preparation. For example, the compositions may be administered to an individual by way of an inhaler, e.g., metered dose inhaler or a dry powder inhaler, an insufflator, a nebulizer or any other conventionally known method of administering inhalable medicaments.

Optionally, the disclosed compositions are in a form suitable for intranasal administration. Such compositions are suitable for delivery into the nose and nasal passages through one or both of the nares and can comprise delivery by a spraying mechanism or droplet mechanism, or through aerosolization.

The disclosed compositions may be suitable for systemic administration to a cardiac cell or to a plurality of cardiac cells of a subject, and/or to a renal cell or to a plurality of renal cells of a subject. If the compositions are used in a method wherein topical pulmonary administration is not used, the compositions may be administered by other means known in the art for example, orally, parenterally (e.g., intravenous injection, intramuscular injection, intraperitoneal injection, or subcutaneous injection), suppository, transdermally or topically to the lungs.

### Example

Influenza virus infection of BALB/c mice induced increased channel-mediated release of the nucleotide ATP into the BALF and elevated BALF ATP contributes to development of lung edema and hypoxemia. In BALB/c mice, influenza causes severe lung damage. Importantly, we have shown that following influenza infection, elevated ATP release into BALF is accompanied by increased activation of A₁-AdoR by the ATP degradation product adenosine. These data indicate that adenosine in the bronchoalveolar fluid (BALE) that was generated in response to influenza plays a pivotal role in mediating lung dysfunction consistent with acute lung injury by activating A₁-AdoR.

Effect of Influenza Infection of Mice on BALF ATP and UTP Content.

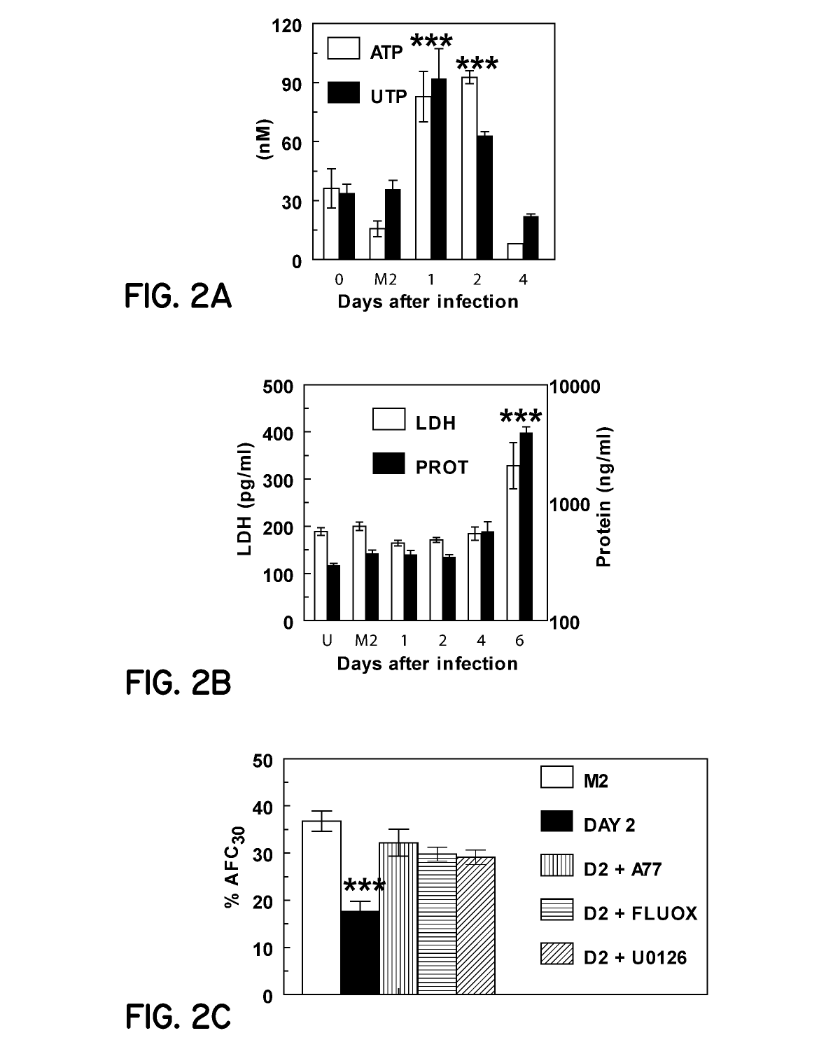

BALB/c mice were infected with 10,000 FFU of mouse-adapted influenza H1N1 virus (A/WSN/33). Control animals were mock-infected with virus diluent (0.1% FCS in saline). Mice (6-8 per group) were euthanized at 2, 4, and 6 days post-infection (d.p.i), and low volume (300 μl) bronchoalveolar lavage (BAL) performed on both lungs. UTP/ATP content was measured in UDP-glucose pyrophosphorylase and luciferin-luciferase assays, respectively. We found that influenza infection, but not mock infection for 2 days (M2), significantly increased BAL ATP and UTP levels (FIG. 2A). Importantly, this release was not temporally associated with increases in BAL markers of epithelial cell death: BAL lactate dehydrogenase (LDH) and protein content (PROT) were not elevated above levels in mock-infected mice until 6 d.p.i. (FIG. 2B). Moreover, we found no histopathologic evidence of any epithelial cell death or sloughing of epithelium until 4 d.p.i. (not shown). Finally, WSN virus-induced suppression of alveolar fluid clearance at 2 d.p.i. was reversed by addition of the de novo pyrimidine synthesis inhibitor, A77-1726 (20 μM), the volume-regulated anion channel inhibitor, fluoxetine (FLUOX) (10 μM), and the ERK MAP kinase inhibitor U0126 (10 μM) to the fluid clearance instillate, to which the animal is only exposed during the 30-min ventilation period over which fluid clearance is measured FIG. 2C).

These data indicate that influenza infection of mice stimulates ERK-induced de novo nucleotide synthesis and volume-regulated anion channel-mediated release of ATP into BALF. ATP release temporally preceeds, and so is a potential inducer but not a consequence of, viral induction of lung injury and epithelial cell death.

Effect of Influenza Infection of Mice on Indices of Lung Function Indicative of Acute Lung Injury.

Current consensus guidelines define acute lung injury as a clinical entity associated with impaired alveolar fluid clearance, an arterial:inspired O₂(PₐO₂:FᵢO₂) ratio <300, increased airway resistance, and decreased lung compliance. Prior to determining the role of adenosine in influenza pathogenesis, we performed a series of functional studies to determine whether influenza-induced lung injury meets these guidelines. We infected C57BL/6 mice with 10,000 FFU of a mouse-adapted influenza virus (A/WSN/33). Outcome measures were evaluated at 2, 4, and 6 d.p.i. in anesthetized, tracheotomized mice, ventilated on 100% O₂ (room air for flexiVent studies). Alveolar fluid clearance was measured by instillation of 300 μl 5% BSA in isosmotic saline into the dependent (left lung) and measuring the change in protein concentration over 30 mins ventilation (with correction for endogenous protein leak). PₐO₂:FᵢO₂ ratio was measured in separate groups of 3-5 mice/timepoint, following 15 mins ventilation on 100% O₂ (FᵢO₂=1), by analysis of a 200 μl carotid aterial blood sample with an i-STAT blood gas analyzer. Finally, lung mechanics were measured by the forced-oscillation technique in mice on a computer-controlled flexiVent piston ventilator.

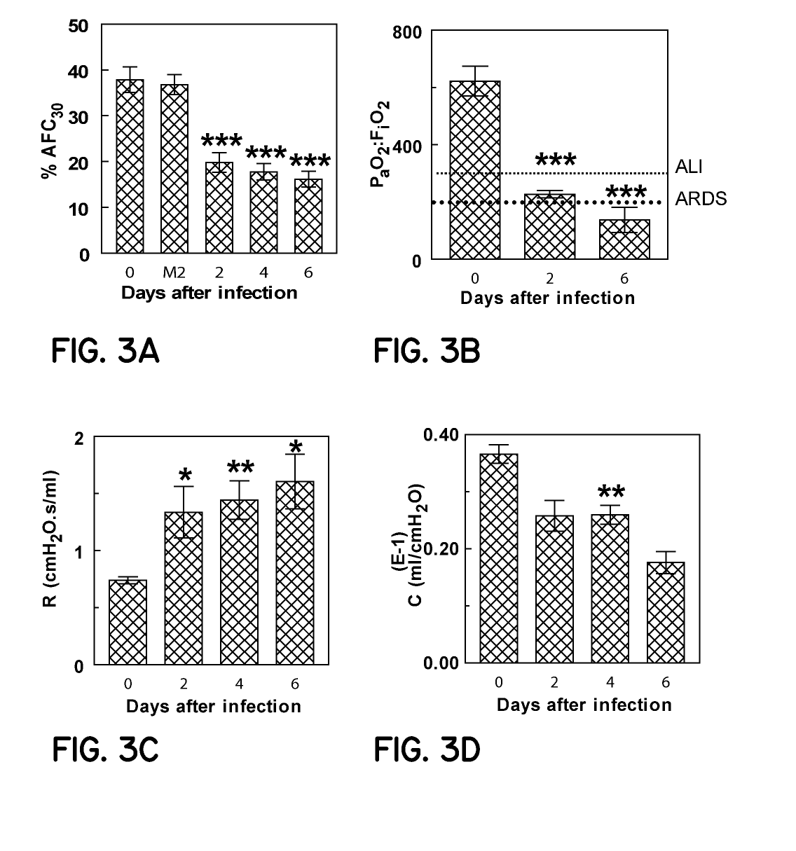

Using these techniques, we found that influenza infection of C57BL/6 mice (n=9-12 per group) results in significant (^{˜}50%) inhibition of alveolar fluid clearance from 2-6 d.p.i. (FIG. 3A). Influenza-induced mice also exhibited impairment of pulmonary gas exchange of a severity consistent with diagnosis of acute lung injury at day 2 (PₐO₂:FᵢO₂<300), and frank acute respiratory distress (ARDS) at day 6 (PₐO₂:FᵢO₂<200; day 4 not yet analyzed) (FIG. 3B). In contrast, uninfected mice maintained a normal PₐO₂:FᵢO₂ ratio (>600) under the same conditions, indicative of normal gas exchange. Finally, total lung resistance (R) was significantly increased from 2 d.p.i. (n−10-12 per group), while lung compliance (C) progressively decreased throughout infection (FIG. 3D).

These data indicate that influenza infection induces lung dysfunction consistent with current definitions of acute lung injury from as early as 2 d.p.i.

Effect of Pharmacologic CD73 Blockade on Acute Lung Injury and Mortality in Influenza-Infected Mice.

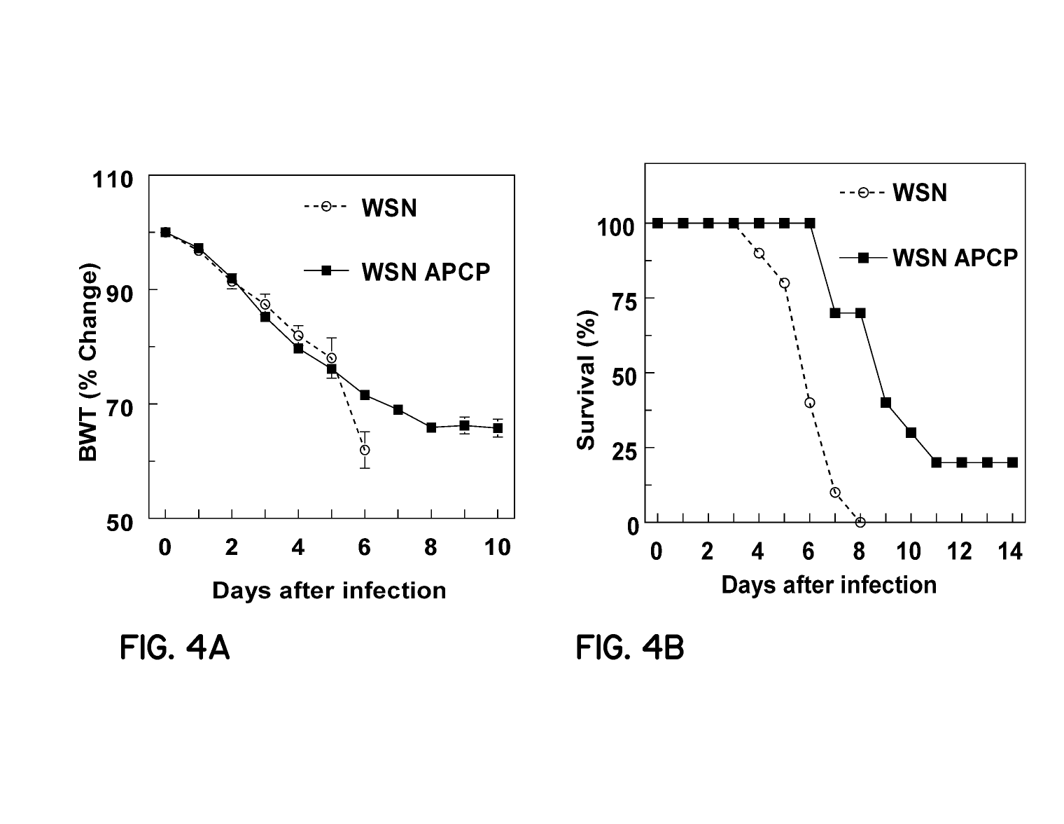

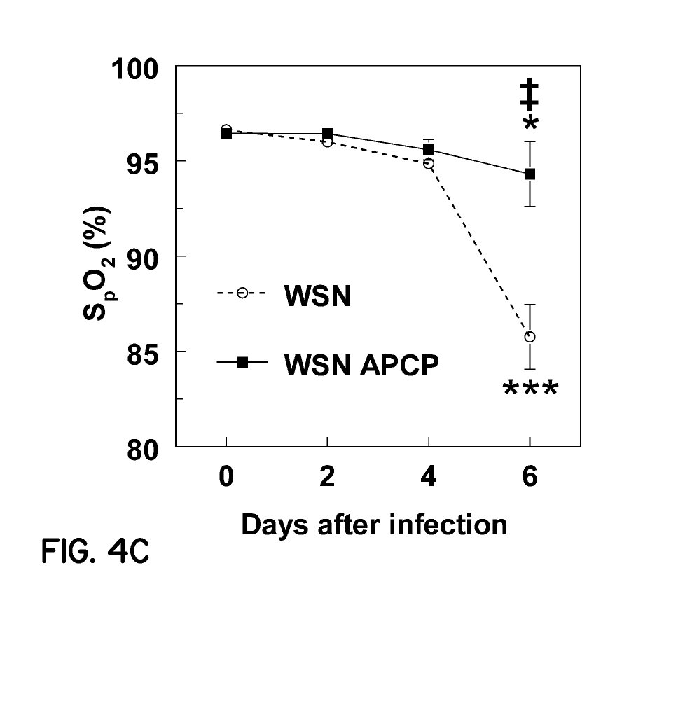

The pharmacologic blockade of CD73 with APCP (5′-(α,β-methylene)diphosphate) reduces BALF adenosine levels and thereby ameliorates acute lung injury in influenza-infected mice. We investigated effects of daily gavage with APCP (20 mg/kg, in 200 μl saline) on body weight, arterial O₂ saturation (SₚO₂; measured in conscious mice with the MouseOx pulse oximetry system) and survival in 2 groups of 10 individually-marked influenza-infected mice, and compared these animals to mock-infected and untreated influenza-infected mice. We found that, while APCP treatment had no significant effect on influenza-induced loss of body weight (BWT; FIG. 4A), it significantly delayed mortality (FIG. 4B) and onset of peripheral hypoxemia, which was present in untreated, influenza-induced mice from 4 d.p.i. and which was severe in this group at 6 d.p.i. (FIG. 4C). In fact, 20% of APCP-treated mice survived infection, whereas all untreated mice died. Importantly, APCP gavage had no effect on lung homogenate virus titers at 2 d.p.i. (not shown).

These data indicate that CD73 blockade improves lung function and ameliorates acute lung injury (impaired gas exchange and altered lung mechanics) in influenza-infected mice.

Effect on A₁-AdoR (Adora1) Gene Knockout on Acute Lung Injury and Cardiac Function in Influenza-Infected Mice.

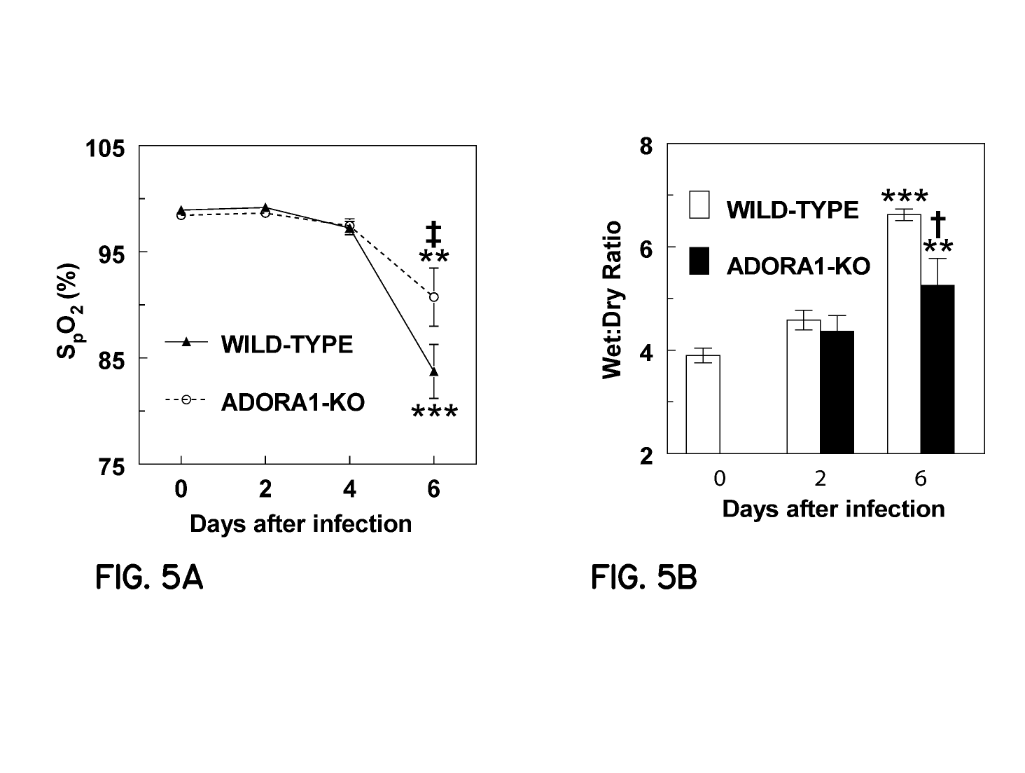

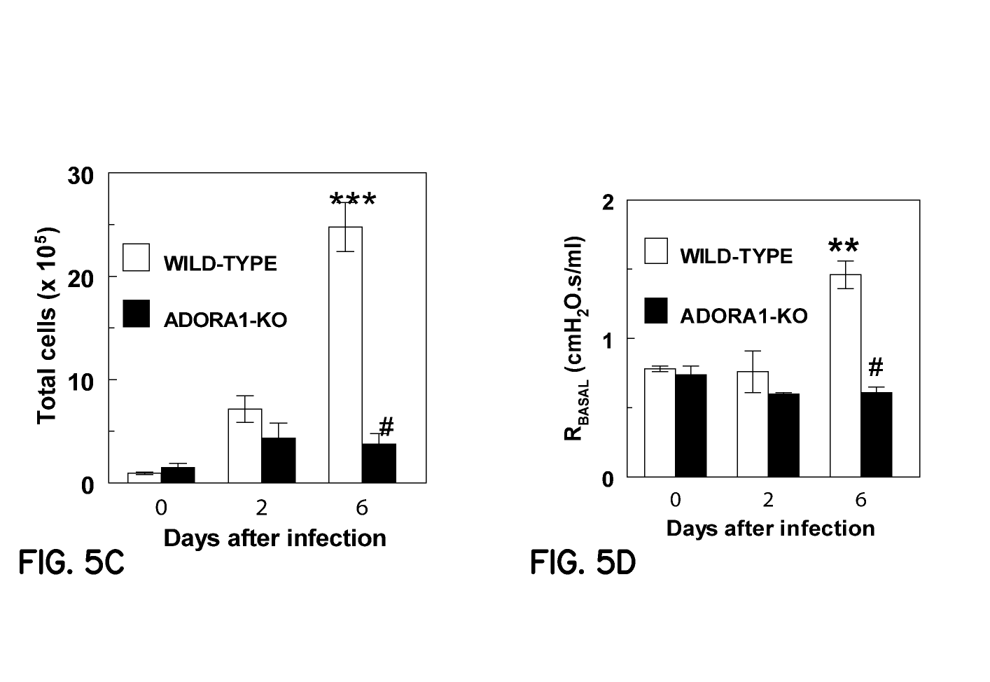

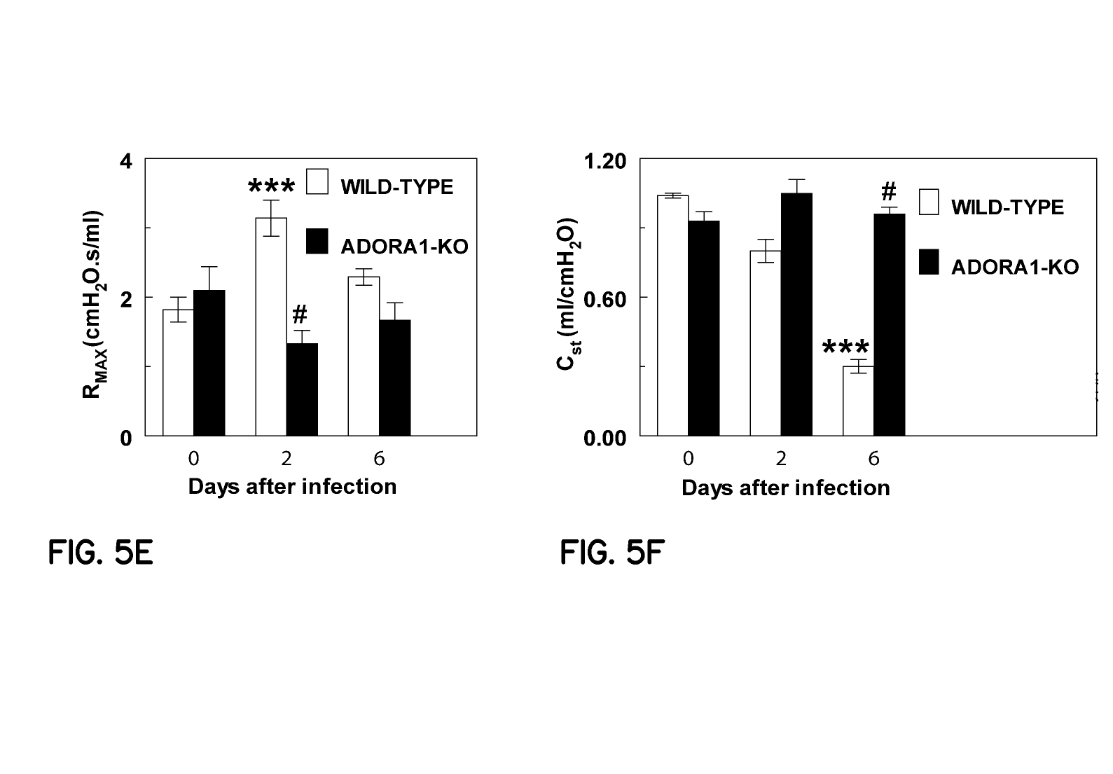

A₁-AdoR activation is pro-inflammatory in influenza infection and A₁-AdoR (adora1) gene-knockout mice exhibit reduced influenza-induced acute lung injury relative to congenic C57BL/6 (wild-type) control mice. C57BL/6 and congenic adora1^{-/-} mice were infected with influenza and the effects of this A₁-AdoR gene knockout on arterial O₂ saturation and heart rate (both measured by pulse oximetry) and lung function indices were determined. We found that, adora1 gene knockout had no significant effect on influenza-induced weight loss (not shown). However adora1^{-/-} mice exhibited significantly reduced peripheral hypoxemia relative to wild-type animals (FIG. 5A). A₁-AdoR gene knockout significantly reduced lung water content (as measured by wet:dry weight ratio) at 6 d.p.i., when lung water is significantly increased in wild-type mice (FIG. 5B)). A₁-AdoR gene knockout also ameliorated pulmonary inflammation since it resulted in a significant reduction in total BAL cell counts at 6 d.p.i. relative to wild-type mice (FIG. 5C). This effect primarily resulted from reduced neutrophil infiltration into the lungs (data not shown). Moreover, A₁-AdoR gene knockout reverse influenza-induced alterations in lung mechanics: adora1-knockout mice were protected from increased basal lung resistance at 6 d.p.i. (FIG. 5D), airway hyperresponsiveness at 2 d.p.i. (FIG. 5E), and reduced static lung compliance at 6 d.p.i. (FIG. 5F), all of which were present in wild-type mice.

These data indicate that genetic deletion of the A₁-AdoR receptor improves pulmonary function and ameliorates acute lung injury in influenza-infected mice. This finding strongly suggests that activation of A₁-AdoR by adenosine plays a role in the pathogenesis of lung dysfunction and acute lung injury in influenza-infected mice.

Effect on Influenza Infection on A₁-AdoR Protein Expression on Murine Alveolar Type II Cells.

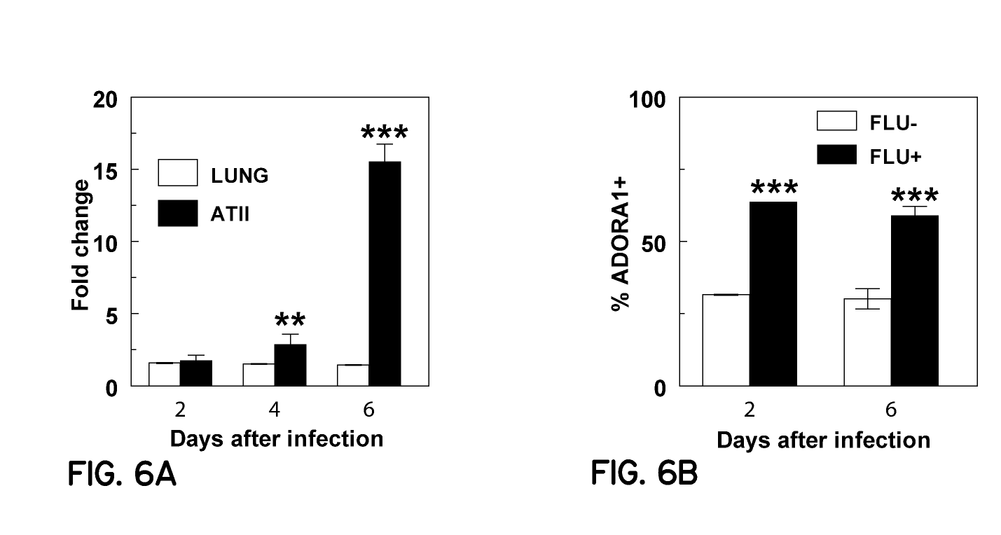

Primary influenza cell targets for infection and viral replication are alveolar epithelial cells, particularly alveolar type II (ATII) cells, although the virus can also infect alveolar macrophages at low levels. Infection of both cell types may result in increased expression of A₁-AdoR on both these influenza-infected cells and, by intercellular signaling, on surrounding uninfected ATII cells and/or alveolar macrophages. This effect will increase pro-inflammatory effects of adenosine on these cell types even in the absence of increased adenosine generation. In addition, infection with influenza may increase A₁-AdoR expression on infiltrating inflammatory cells, which traffic to the lungs in response to inflammatory signals (such as cytokines, chemokines, and adenosine itself) that are released in response to influenza infection. Infiltrating monocytes, neutrophils and lymphocytes can all express A₁-AdoR and expression levels on these cell types can therefore be increased following infection, irrespective of the infection status of individual infiltrating cells. C57BL/6 mice were infected with influenza ATII cells were isolated from mouse lung at 2 and 6 d.p.i. and influenza effects on adora1 gene (mRNA) and A₁-AdoR protein expression were assessed by real-time RT-PCR and flow cytometry, respectively. Influenza infection resulted in increased ATII cell adora1 gene transcription (elevated mRNA levels) at 6 d.p.i. in homogenates of >95% pure ATII cell preparations, but not in whole lung homogenates (FIG. 6A). Moreover, following influenza infection, a significantly higher percentage of influenza-infected ATII cells were A₁-AdoR-positive than uninfected ATII cells from the same lungs (FIG. 6B).

These data indicate that influenza infection increases A₁-AdoR expression on ATII cells, which will increase responsiveness of these cells to adenosine even in the absence of increased intra-alveolar adenosine generation.

Effect on Systematic Administration of the A1-AdoR Antagonist DPCPX on Acute Lung Injury and Mortality in Influenza-Infected Mice.

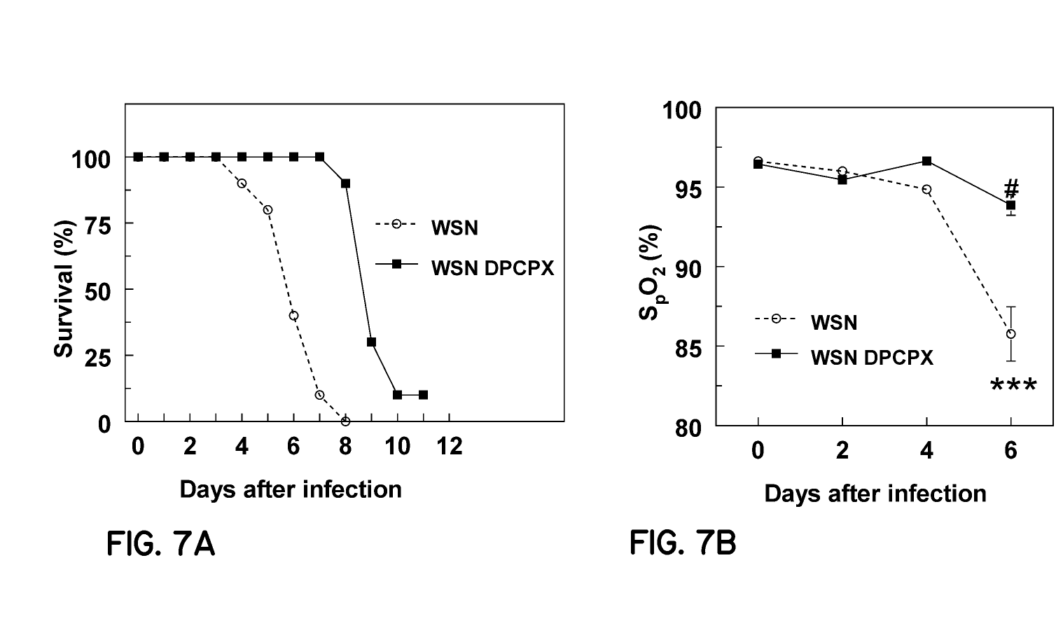

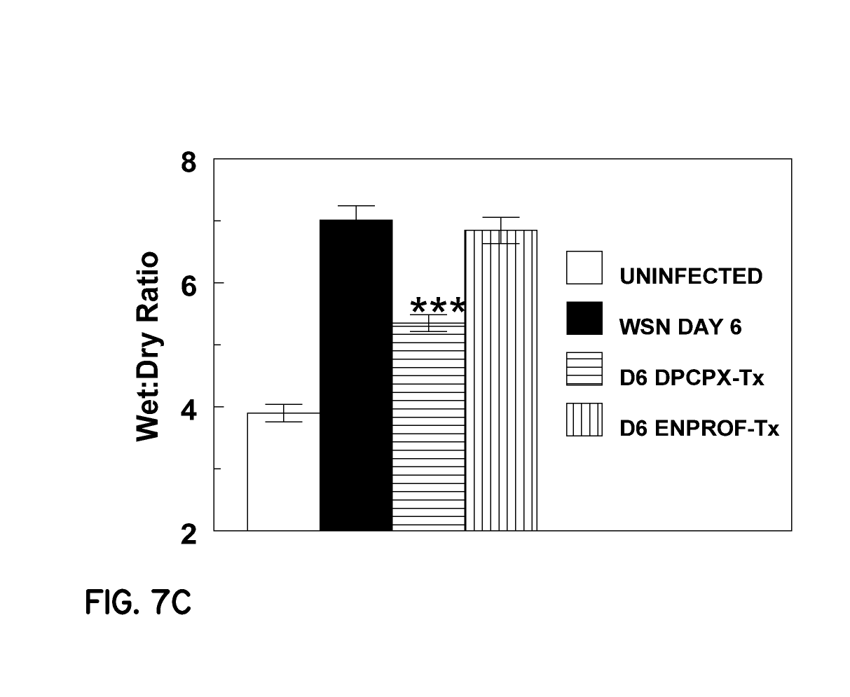

A₁-AdoR activation is pro-inflammatory in influenza infection and pharmacologic blockade of A₁-AdoR with the prototypical A₁-AdoR antagonist DPCPX (8-Cyclopentyl-1,3-diproopylxanthine) ameliorates adenosine-induced acute lung injury in influenza-infected mice. Influenza-infected mice were treated with DPCPX (1 mg/kg/day), administered by implanted osmotic minipump (Alzet). The effects of this A₁-AdoR antagonist on body weight, arterial O₂ saturation (measured by pulse oximetry) and survival were investigated in 2 groups of 10 individually-marked influenza-infected mice. For some outcome measures, we also evaluated the effect of daily administration of the A_{2b}-AdoR antagonist enprofylline (4 mg/kg I.P., in 100 μl 10% ethanol in saline; EMD Biosciences), to determine whether A_{2b}-AdoR blockade also modulates influenza outcomes. We found that, like APCP treatment, neither DPCPX nor enprofylline had any significant effect on influenza-induced weight loss. However (and also like APCP), DPCPX treatment significantly delayed mortality (FIG. 7A) and onset of peripheral hypoxemia (FIG. 7B). 10% of DPCPX-treated mice survived infection. Neither DPCPX nor enprofylline had any effect on lung homogenate virus titers at 6 d.p.i. (not shown). DPCPX, but not enprofylline treatment, also ameliorated pulmonary inflammation since it resulted in a significant reduction in total BAL cell counts at 6 d.p.i. (not shown). Finally, DPCPX treatment also significantly reduced lung water content (as measured by wet:dry weight ratio) at 6 d.p.i. (when lung water is significantly increases). In contrast, enprofylline treatment had no such effect (FIG. 7C).

These data indicate that systematic administration of the A₁-AdoR antagonist DPCPX improves lung function and ameliorates acute lung injury in influenza-infected mice. In contrast, the A_{2b}-AdoR antagonist enprofylline has no detectable effect on influenza pathogenesis. This finding strongly suggests that activation of A₁-AdoR, but not A_{2b}-AdoR, by adenosine plays a role in the pathogenesis of acute lung injury in influenza-infected mice.

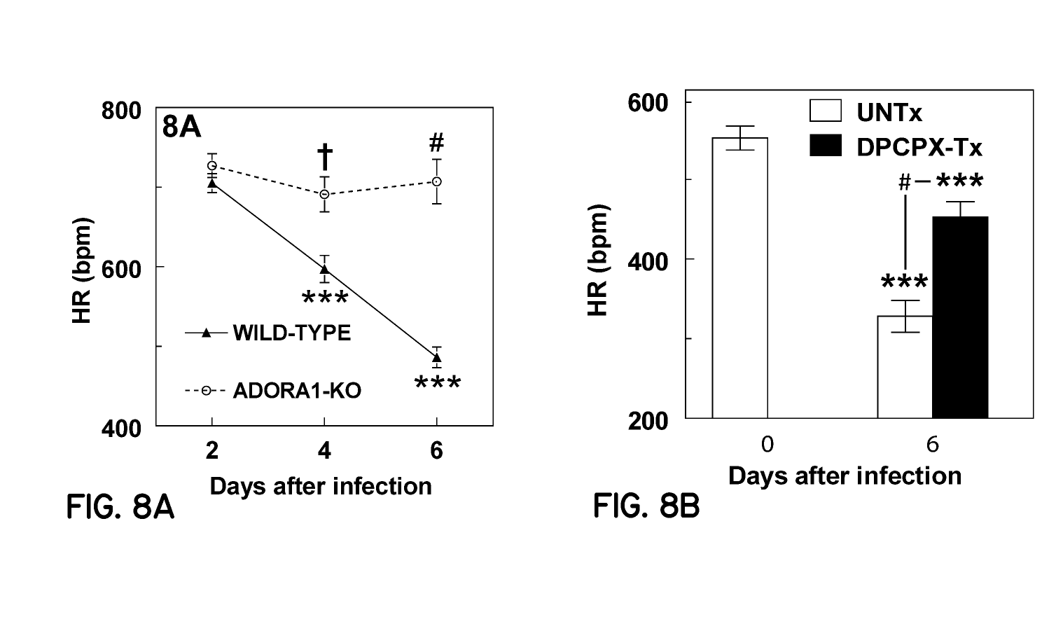

A₁-AdoR (adora1) gene knockout or pharmacologic blockade of A₁-AdoR with the prototypical A₁-AdoR antagonist DPCPX (8-Cyclopentyl-1,3-dipropylxanthine) ameliorates adenosine-induced cardiac dysfunction in influenza-infected mice. Infection of BALB/c mice with influenza A/WSN/33 (10,000 PFU/mouse) for 6 days results in bradycardia that is absent in adora1^{-/-} mice (FIG. 8A) and also reversed by systemic treatment with the A₁-AdoR antagonist DPCPX (FIG. 8B), but no evidence of myocarditis or cardiac influenza infection (not shown).

These data indicate that A₁-AdoR (adora1) gene knockout or systematic administration of the A₁-AdoR antagonist DPCPX improves cardiac function in influenza-induced mice.

The data for this example were generated with the following methods.

Preparation of Viral Inocula.

Influenza A/WSN/33 (H1N1) virus (WSN virus; a mouse-adapted H1N1 human influenza strain, which is pneumotropic following intranasal inoculation) was grown in Madin-Darby canine kidney cells and its infectivity assayed by fluorescent-focus assay 24 hrs after inoculation of the NY3 fibroblast cell line (derived from STAT1^{-/-} mice).

Animals.

8-12 week-old C57BL/6 mice and congenic adora1^{-/-} mice of either sex, maintained in autoclaved microisolators, were used. The pathogen-free status of all animals were monitored by culture for mycoplasmal, viral, fungal, and bacterial pathogens (Charles River Biotechnical Services, Spencerville, Ohio). Animals were given sterile autoclaved food and water ad libitum, and monitored daily.

Infection of Mice with Influenza.

Mice were infected intranasally with 50 μl influenza A/WSN/33 under 3% isoflurane anesthesia. Mock-infected animals received 50 μl of virus diluent (PBS with 0.1% BSA). In some experiments, mice were individually marked and weighed daily.

Measurement of Peripheral Blood Arterial Oxygen Saturation and Heart Rate.

Saturations and heart rates were measured in individually-marked conscious mice with the MouseOx system (Starr Life Sciences Corp., Allison Park, Pa.).

Alveolar Fluid Clearance Measurements.

Mice were anesthetized with valium (1.75 mg/100 g weight) followed by ketamine (45 mg/100 g weight) I.P., tracheotomized, and a trimmed sterile 18-g catheter inserted caudally into the tracheal lumen. Following administration of pancuronium (0.08 μg/kg I.P.), each mouse was placed on a Deltaphase® isothermal heating pad (Braintree Scientific, Braintree, Mass.), and ventilated with a Model 687 volume-controlled mouse ventilator (Harvard Apparatus, Holliston, Mass.), on 100% O₂, at 160 breaths/min. 300 μl of 5% BSA/saline was instilled into the dependent (left) lung. After 30 minutes ventilation, instilled fluid was aspirated to measure protein content and calculate fluid clearance rate.

Measurement of Arterial Blood Gases and Calculation of PₐO₂:FᵢO₂ ratio. Mice were anesthetized as for AFC procedures, and ventilated for 10 mins on 100% O₂ (FᵢO₂=1.0). A sample of arterial blood was then taken from the abdominal aorta and PₐO₂ measured on an Abbott-1-STAT blood gas analyzer.

Assessment of Lung Function.

Lung function was measured by the forced-oscillation technique. Each mouse was anesthetized and tracheotomized as for AFC studies, then mechanically ventilated on a computer-controlled piston ventilator (flexiVent, SciReq; Montreal, Canada), with the following parameters: V_{T} 8 ml/kg; frequency 150 breaths/min; FᵢO₂-0.21. Following two total lung capacity maneuvers to standardize volume history, pressure and flow data were collected during a series of standardized volume perturbation maneuvers. These data are used to calculate P-V loops and total lung resistance (R) and elastance (E) using the single-compartment model.

Euthanasia of Mice.

Following anesthesia for pulmonary function assays, mice were euthanized by exsanguination. Blood was collected by axillary section into tubes containing 3% EDTA, centrifuged at 9,400 g for 10 mins, and plasma stored at −80° C. for subsequent analysis.

Measurement of Lung Wet:Dry Weight.

The right lung was removed weighed, dried in an oven at 55° C. for 5 days, then reweighed. Wet-to-dry weight ratio provides an index of intrapulmonary fluid accumulation.

Bronchoalveolar Lavage and Assays of Lavage Fluid.

Following removal of the left lung, the right lung was lavaged in situ with 0.5 ml of sterile saline. Lavagates were centrifuged and the cells gently resuspended in sterile saline. Numbers of viable alveolar macrophages, lymphocytes, and polymorphonuclear cells were calculated from total leukocytes (counted using a hemocytometer with 0.4% trypan blue exclusion to assess viability) and differential counts of Diff-Quik-stained cytocentrifuge preparations. Supernatants were stored at −80° C. BAL protein and LDH content were determined by standard colorimetric assays.

Detection of Bronchoalveolar Lavage Fluid Nucleotides.

Lungs from euthanized mice were lavaged in situ with 300 μl of sterile saline containing the ADA inhibitor erythro-9-(2-hydroxy-3-nonyl)adenine hydrochloride (EHNA; 2.5 μM) and the nucleoside transport inhibitor dipyridamole (250 μM) (50). BAL fluid was centrifuged (800 rpm, 5 mins at 4° C.) and the supernatant boiled for 2 mins to inactivate endogenous nucleotidases. Nucleotide analysis was then be performed by HPLC.

Isolation and Flow Cytometric Analysis of Alveolar Type II Cells.

ATII cells were isolated from C57BL/6 mice using the method of Corti et al. Following euthanasia, the heart was exposed by thoracotomy, the right ventricle opened, and the pulmonary circulation flushed clear with sterile saline. The trachea was then cannulated with a trimmed 18-g intravenous catheter. 2.5 ml dispase (BD) was then injected into the lungs via the tracheal cannula, followed by 0.45 ml of 1% low melting point agarose in dIH₂O, heated to 45° C. (to prevent isolation of Clara cells and upper airway epithelial cells). After cooling the mouse thorax with ice for 2 mins, the heart was excised, and the lungs removed from the chest cavity, rinsed with sterile saline, and placed in 5 ml dispase to digest at room temperature for 45 mins. Lung tissue was then teased apart in 7.5 ml of 0.01% DNase I in DMEM. The resulting cell suspension was sequentially filtered through sterile 100 μm, 40 μm, and 25 μm nylon mesh, centrifuged, washed in DMEM/10% FBS, and resuspended in 80 μl staining buffer/10⁷ cells. Cells were then incubated at 4° C. for 15 mins with rabbit anti-prosurfactant-C pAb (AB3786MI, 10 μl/10⁷ cells, Millipore, Bradford, Ill.), followed by a second 15-min incubation at 4° C. in the presence of anti-rabbit MACS® MicroBeads (Miltenyi Biotec Inc., Auburn, Calif.), then washed. ATII cells were positively selected by passing the treated cell suspension through an autoMACS™ cell separator. Eluted ATII cells were pelleted by centrifugation, resuspended in DMEM/10% FBS, and counted in a hemocytometer. Purity of isolated ATII cell preparations was assessed by Papanicolau staining and flow cytometry on a FACScalibur dual laser flow cytometer following immunostaining with an antibody to surfactant protein C(SP-C). An APC LYNX®-conjugated mouse-specific polyclonal antibody was used to evaluate expression of A₁-AdoR.

Real-Time PCR of Purified ATII Cells.

Total RNA was isolated from 30 mg of fresh lung tissue per mouse, or from isolated FACS-purified cells using the TRIzol® reagent (Invitrogen), according to a standard protocol. Final RNA quality was assessed by comparing 28S and 18S rRNAs after electrophoresis through 1.5% agarose/2.2 mM formaldehyde gel, under UV light with ethidium bromide staining. Samples exhibiting RNA degradation were discarded. cDNAs were generated by reverse transcription, using the High Capacity cDNA RT kit (Applied Biosystems). Negative control reactions (for genomic DNA contamination) were performed in the absence of reverse transcriptase. Gene expression was determined using the TagMan® Fast Real-Time Gene Expression Master Mix and TagMan® Gene Expression Assay pre-designed, validated, mouse-specific primer pairs for the adora1 gene (both Applied Biosystems) in a 96-well plate format on a Roche LightCycler® 480 Real-Time PCR system (Roche Diagnostics, Indianapolis, Ind.). cDNA prepared from each animal were assayed at 20 ng/μl in triplicate for the adora1 gene, together with one reaction for gapdh. After PCR, a dye fluorescence threshold within the exponential phase of the reaction was set separately for the target gene (T_{g}) and the endogenous reference (Eᵣ; gapdh). The cycle number at which each amplified product crosses the set threshold (C_{T} value) was determined and the amount of T_{g} normalized to Eᵣ by subtracting the Eᵣ C_{T} from the T_{g} C_{T} (ΔC_{T}). Relative mRNA expression was calculated by subtracting the mean ΔC_{T} of control samples from mean ΔC_{T} of the treated samples (ΔΔC_{T}). The amount of T_{g} mRNA was then calculated using the formula 2-ΔΔC_{T}.

Biosafety Precautions.

Biosafety Level 2 practices were employed when working with influenza-infected cells or animals. All procedures using infected cells or tissues were performed in a Class II biological safety hood to avoid generation of potentially infectious aerosols. Waste materials were autoclaved prior to disposal.

Statistical Analyses.

Descriptive statistics (mean and standard error) were calculated using Instat software (GraphPad). A two-sample t-test was used for two-group comparisons. For more than two groups, ANOVA were used to assess significance, with a post hoc Tukey test to determine which of the group(s) is different from the rest if significance is found. Association was tested using Pearson's correlation coefficient. All data were reported as mean±S.E.M. P<0.05 was considered statistically significant.

What is claimed is:

1. A method of treating a subject for a pulmonary, cardiac, and/or renal dysfunction resulting from an influenza infection comprising:
administering an effective amount of a pharmaceutical composition to disrupt an adenosine receptor pathway in the subject to treat the pulmonary, cardiac, or renal dysfunction.

2. The method of claim 1 wherein the adenosine receptor pathway is in at least one of the lung tissue, the cardiac tissue, or the renal tissue of the subject.

3. The method of claim 1 wherein the adenosine receptor pathway is the A₁-adenosine receptor pathway.

4. The method of claim 1 wherein the disruption of the adenosine receptor pathway includes at least one of the decreasing the synthesis of ATP, decreasing the release of ATP from a cell, decreasing the conversion of ATP to adenosine, decreasing the expression of the adenosine receptor, decreasing the activation of the adenosine receptor, and increasing the clearance of adenosine from the extracellular space.

5. The method of claim 1 wherein the pharmaceutical composition includes at least one of an adenosine receptor antagonist, an inhibitor of adenosine receptor gene expression, an inhibitor of adenosine receptor protein expression, a disruptor of pyrimidine synthesis, an ATP hydrolysis inhibitor, an inhibitor of cd39 gene expression, an inhibitor of CD39 protein expression, an inhibitor of cd73 gene expression, an inhibitor of CD73 protein expression, a VRAC inhibitor, a Rho kinase inhibitor, an adenosine deaminase activator, and an equilibrative nucleotide transporter activator.

6. The method of claim 5 wherein the adenosine receptor antagonist is an A₁-adenosine receptor antagonist.

7. The method of claim 6 wherein the A₁-adenosine receptor antagonist includes at least one of 8-cyclopentyl-1,3-dipropylxanthine (DPCPX), L-97-1, SLV320, rolofylline, and cyclopentyltheophylline.

8. The method of claim 5 wherein the inhibitor of adenosine receptor gene or protein expression is an inhibitor of expression of the gene encoding the A₁-adenosine receptor, adora1.

9. The method of claim 5 wherein the inhibitor of adenosine receptor gene or protein expression includes at least one of small inhibitory RNAs directed against A₁-adenosine receptor mRNA, microRNAs directed against A₁-adenosine receptor mRNA, and vector-mediated or other constructs designed to specifically induce inactivation of adora1 gene transcription and translation.

10. The method of claim 5 wherein the disruptor of pyrimidine synthesis includes at least one of A77-1726 or U0126.

11. The method of claim 5 wherein the ATP hydrolysis inhibitor inhibits the activity of at least one of CD39 and CD73.

12. The method of claim 5 wherein the ATP hydrolysis inhibitor includes at least one of polyoxymetate-1 (“POM-1”), ARL67156, 5′-(a,b-methylene)diphosphate (“APCP”) or small inhibitory RNA molecules directed against the mRNA of at least one of CD39 or CD73.

13. The method of claim 5 wherein the VRAC inhibitor includes at least one of fluoxetine, clomiphene, verapamil, 5-nitro-2-(3-phenylpropylamino) benzoic acid (“NPPB”), R(+)-IAA 94 (R(+)-([6,7-dichloro-2-cyclopentyl-2,3-dihydro-2-methyl-1-oxo-1H-inden-5-yl]-oxy)acetic acid 94), and tamoxifen.

14. The method of claim 5 wherein the at least one of the inhibitor of cd39 gene expression or the inhibitor of CD39 protein expression includes at least one of a hypoxia inducible factor inhibitor-1 (“HIF-1”) inhibitor, small inhibitory RNAs directed against CD39 mRNA, microRNAs directed against CD39 mRNA, and vector-mediated or other constructs designed to specifically induce inactivation of cd39 gene transcription and/or translation.

15. The method of claim 5 wherein the inhibitor of cd73 gene or protein expression includes at least one of small inhibitory RNAs directed against CD73 mRNA, microRNAs directed against CD73 mRNA, and vector-mediated or other constructs designed to specifically induce inactivation of cd73 gene transcription and CD73 protein translation.

16. The method of claim 14 wherein the HIF1 inhibitor includes at least one of 3-(5′-hydroxymethyl-2′-furyl)-1-benzyl indazole (“YC-1”) and PX-478.

17. The method of claim 5 wherein the Rho kinase inhibitor includes at least one of (S)-(+)-2-Methyl-1-[(4-methyl-5-isoquinolinyl)sulfonyl]homopiperazine (“H-1152”), N-(4-Pyridyl)-N′-(2,4,6-trichlorophenyl)urea (“NNU”), 3-(4-Pyridyl)-1H-indole (“Rockout”), and N-(4-(1H-pyrazol-4-yl)phenyl)-2,3-dihydrobenzo[b][1,4]dioxine-2-carboxamide (“pyrazol carboxamide”).

18. The method of claim 5 wherein the adenosine deaminase activator includes at least one of 2′-deoxycoformycin or 2-N-methyl-2,4-diazacycloheptanone.

19. The method of claim 5 wherein the ENT activator includes at least one of a protein kinase C (“PKC”) activator or a HIF-1 inhibitor.

20. The method of claim 19 wherein the PKC activator includes phorbol 12-myristate 13-acetate (“PMA”).

21. The method of claim 19 wherein the HIF1 inhibitor includes at least one of YC-1 and PX-478.

22. The method of claim 1 wherein the pharmacological formulation is administered by at least one of inhalation, injection, oral ingestion, suppository insertion, and transdermally.

23. A method of treating a subject for a pulmonary, cardiac, and/or renal dysfunction resulting from a viral infection comprising:
administering an effective amount of a pharmaceutical composition to disrupt an adenosine receptor pathway in the subject to treat the pulmonary, cardiac, or renal dysfunction.

24. The method of claim 23 wherein the adenosine receptor pathway is in at least one of the lung tissue, the cardiac tissue, or the renal tissue of the subject.

25. The method of claim 23 wherein the adenosine receptor pathway is the A₁-adenosine receptor pathway.

26. The method of claim 23 wherein the disruption of the adenosine receptor pathway includes at least one of the decreasing the synthesis of ATP, decreasing the release of ATP from a cell, decreasing the conversion of ATP to adenosine, decreasing the expression of the adenosine receptor, decreasing the activation of the adenosine receptor, and increasing the clearance of adenosine from the extracellular space.

27. The method of claim 23 wherein the pharmaceutical composition includes at least one of an adenosine receptor antagonist, an inhibitor of adenosine receptor gene expression, an inhibitor of adenosine receptor protein expression, a disruptor of pyrimidine synthesis, an ATP hydrolysis inhibitor, an inhibitor of cd39 gene expression, an inhibitor of CD39 protein expression, an inhibitor of cd73 gene expression, an inhibitor of CD73 protein expression, a VRAC inhibitor, a Rho kinase inhibitor, an adenosine deaminase activator, and an equilibrative nucleotide transporter activator.

28. The method of claim 27 wherein the adenosine receptor antagonist is an A₁-adenosine receptor antagonist.

29. The method of claim 28 wherein the A₁-adenosine receptor antagonist includes at least one of DPCPX, L-97-1, StV320, rolofylline, and cyclopentyltheophylline.

30. The method of claim 27 wherein the inhibitor of adenosine receptor gene or protein expression is an inhibitor of expression of the gene encoding the A₁-adenosine receptor, adora1.

31. The method of claim 27 wherein the inhibitor of adenosine receptor gene or protein expression includes at least one of small inhibitory RNAs directed against A₁-adenosine receptor mRNA, microRNAs directed against A₁-adenosine receptor mRNA, and vector-mediated or other constructs designed to specifically induce inactivation adora1 gene transcription and translation.

32. The method of claim 27 wherein the disruptor of pyrimidine synthesis includes at least one of A77-1726 or U0126.

33. The method of claim 27 wherein the ATP hydrolysis inhibitor inhibits the activity of at least one of CD39 and CD73.

34. The method of claim 27 wherein the ATP hydrolysis inhibitor includes at least one of POM-1, ARL67156, APCP, or small inhibitory RNA molecules directed against the mRNA of at least one of CD39 or CD73.

35. The method of claim 27 wherein the VRAC inhibitor includes at least one of fluoxetine, clomiphene, verapamil, NPPB, R(+)-IAA 94 (R(+)-([6,7-dichloro-2-cyclopentyl-2,3-dihydro-2-methyl-1-oxo-1H-inden-5-yl]-oxy)acetic acid 94), and tamoxifen.

36. The method of claim 27 wherein the at least one of the inhibitor of cd39 gene expression or the inhibitor of CD39 protein expression includes at least one of a HIF-1 inhibitor, small inhibitory RNAs directed against CD39 mRNA, microRNAs directed against CD39 mRNA, and vector-mediated or other constructs designed to specifically induce inactivation of cd39 gene transcription and/or translation.

37. The method of claim 27 wherein the inhibitor of cd73 gene or protein expression includes at least one of small inhibitory RNAs directed against CD73 mRNA, microRNAs directed against CD73 mRNA, and vector-mediated or other constructs designed to specifically induce inactivation of cd73 gene transcription and CD73 protein translation.

38. The method of claim 27 wherein the HIF1 inhibitor includes at least one of YC-1 and PX-478.

39. The method of claim 27 wherein the Rho kinase inhibitor includes at least one of H-1152, N-NNU, Rockout, and pyrazol carboxamide.

40. The method of claim 27 wherein the adenosine deaminase activator includes at least one of 2′-deoxycoformycin or 2-N-methyl-2,4-diazacycloheptanone.

41. The method of claim 27 wherein the ENT activator includes at least one of a PKC activator or a HIF-1 inhibitor.

42. The method of claim 41 wherein the PKC activator includes PMA.

43. The method of claim 41 wherein the HIF1 inhibitor includes at least one YC-1 and PX-478.

44. The method of claim 23 wherein the pharmacological composition is administered by at least one of inhalation, injection, oral ingestion, suppository insertion, and transdermally.

45. The method of claim 23 wherein the viral infection is an infection by a virus from at least one of Orthomyxviridae, Paramyxoviridae, Togaviridae, Hantaviridae, Rhinoviridae, Coronoviridae, Herpesviridae, Adenoviridae, and Filoviridae.

46. The method of claim 23 wherein the viral infection is an infection by at least one of an influenza A virus, an influenza B viruses, H5N1 virus, H1N1 virus, respiratory syncytial virus, Hendra virus, Nipah virus, rubella virus, Sin Nombre virus, Epstein Barr virus, and cytomegalovirus.
